(19) United States
(12) Patent Application Publication
(10) Pub. No.: US 2010/0183782 A1
(43) Pub. Date: Jul. 22, 2010

(54) SYSTEM, METHOD AND APPARATUS FOR CONTROLLING MICROBIOLOGICAL CONTAMINATION IN COMMERCIAL FREEZE DRYERS USING UV ENERGY AND PHOTOHYDROIONIZATION CELL TECHNOLOGY

(75) Inventors: James L. Marsden, Manhattan, KS (US); Marie Moody, New Berlin, WI (US)

Correspondence Address: SHUTTS & BOWEN, LLP, 100 S. ASHLEY DRIVE, SUITE 1500, TAMPA, FL, 33602, US

(73) Assignee: Stella & Chewy's, LLC, Muskego, WI (US)

(21) Appl. No.: 12/690,695

(22) Filed: Jan. 20, 2010

Related U.S. Application Data
(60) Provisional application No. 61/145,835, filed on Jan. 20, 2009.
(60) Provisional application No. 61/145,822, filed on Jan. 20, 2009.

(51) Int. Cl. A23L 3/3409; A23B 4/14; B01J 19/08; A61L 2/20

(52) U.S. Cl. 426/236; 422/29; 422/186.3

(57) ABSTRACT

The system, method and apparatus involves the control of microbiological contamination of commercial freeze dryers using advanced oxidation gases produced by Photohydroionization (PHI) Cell technology. The PHI Cell system produces hydro peroxides, super oxide ions, ozonide ions, hydroxides and other oxidative gases. These compounds act as anti-microbial agents and systematically inactivate bacteria, viruses, yeast and mold in the air and on surfaces inside commercial freeze dryers.

### CROSS-REFERENCE TO RELATED APPLICATIONS

This application relates to the same subject matter as co-pending provisional patent application Ser. No. 61/145,835, filed by the same applicant on Jan. 20, 2009, and co-pending provisional patent application Ser. No. 61/145,822, filed by the same applicant on Jan. 20, 2009. This application claims the Jan. 20, 2009 filing date as to the common subject matter.

### BACKGROUND OF THE INVENTION

1. Field of Invention

The present invention relates to a system, method and apparatus for the control of microbiological contamination. It applies particularly to control in commercial freeze dryers, and will be described with particular reference thereto.

2. Background of Invention

The current systems, methods and equipment for cleaning and sanitizing commercial freeze dryers, especially those used in the processing of food for humans and animals, involve the application of detergents and liquid sanitizers. This application may be done on a daily basis or less frequently.

These traditional methods and equipment do not involve the use of plasma, gas or vapor based anti-microbial compounds. When foam detergents and/or liquid sanitizers are used, some areas in an environment or on a surface of a particular piece of equipment may not be treated during the cleaning or sanitation process. The failure may be a function of ability of the product used to cover the entire surface. Also, the application is often performed by people, and human errors may be made in the application of the detergents and sanitizers. If the environment around, on or within a piece of equipment becomes contaminated during operations and is not properly treated afterwards, microorganisms may colonize inside the equipment and act as a vector of contamination during the freeze drying process. In some cases wherein cleaning and sanitizing occurs infrequently, the potential for recontamination or increased contamination may exist for long periods of time.

The present invention provides a system, method and apparatus using Photohydroionization™, or PHI™ technology. PHI is an advanced oxidation technology developed and owned by RGF Environmental Group, Inc. It is a broad spectrum high intensity UV light targeted on a catalyst ultraviolet (UV) target in a low-level ozone and moist atmosphere. This creates an advanced oxidation process providing friendly oxidizers, or very safe and aggressive oxidizers that revert back to oxygen and hydrogen. PHI™ technology has been successfully used in water, air, food, laundry and grease applications.

A PHI Cell™ is a product of advanced oxidation technology developed by RGF Environmental Group, Inc. A PHI Cell comprises an assembly including a high-intensity broad spectrum UV bulb or tube (approximately 100-300 nm) in a hydrated catalytic matrix cell. Low-level ozone is produced in the cell, the majority of which is converted into airborne hydro peroxides, super oxide ions, ozonide ions and hydroxides. The UV bulb or tube is encased by a protector such as a protective tube to help prevent any glass or mercury breakage/leakage. Also, the entire assembly may itself be encased in a protective cell.

PHI Cell technology is used in the present invention for enhanced control of microbiological contamination in food processing equipment such as commercial freeze dryers.

Where the system of the invention is mounted external to the unit, the equipment may be cleaned and sanitized either through traditional means or by using a Clean-in-Place (CIP) system. The PHI technology may act as an adjunct to routine cleaning and sanitation. The combination of proper cleaning and sanitation and intermittent and/or continuous treatment with the PHI technology will substantially reduce the risk of recontamination during the freeze drying process.

### SUMMARY OF THE INVENTION

In accordance with one aspect of the invention, a system, method and apparatus for the control of microbiological contamination is described herein. It applies particularly to control in commercial freeze dryers.

The system and method involve the decontamination of commercial freeze dryers using advanced oxidation gases produced by PHI technology. The PHI technology system produces into the external environment hydro peroxides, super oxide ions, ozonide ions, hydroxides and other oxidative gases. These compounds act as anti-microbial agents and systematically inactivate bacteria, viruses, yeast and mold in the air and on surfaces inside commercial freeze dryers.

The system, method and apparatus described above eliminate or reduce biological hazards, including microbiological pathogens such as those found in human and animal food processing equipment such as commercial freeze dryers. This helps prevent potential recontamination of food products, vitamins and pharmaceuticals during processes such as the freeze drying process.

This system, method and apparatus allow for the introduction of anti-microbial compounds in gas or vapor form which address all internal surfaces and air space inside commercial freeze dryers. For example, freeze dryers are inherently difficult to clean and sanitize and as a result, pose a risk for environmental contamination of food, vitamins and pharmaceuticals during the freeze drying process. The oxidative gases also help prevent the colonization of microorganisms inside equipment.

The system, method and apparatus may be applied intermittently during cleaning and sanitation done at regular or irregular intervals or continuously during operation of the freeze dryer.

The system, method and apparatus involves the installation of a PHI Cell on commercial freeze dryers. The installation may be external or internal. Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating preferred embodiments and are not to be construed as limiting the invention.

FIG. 1 is a schematic diagram of one embodiment of the system of the invention.

FIG. 2 is a schematic diagram of another embodiment of the system of the invention.

FIG. 3 is a schematic diagram of another embodiment of the system of the invention.

### DETAILED DESCRIPTION OF THE INVENTION

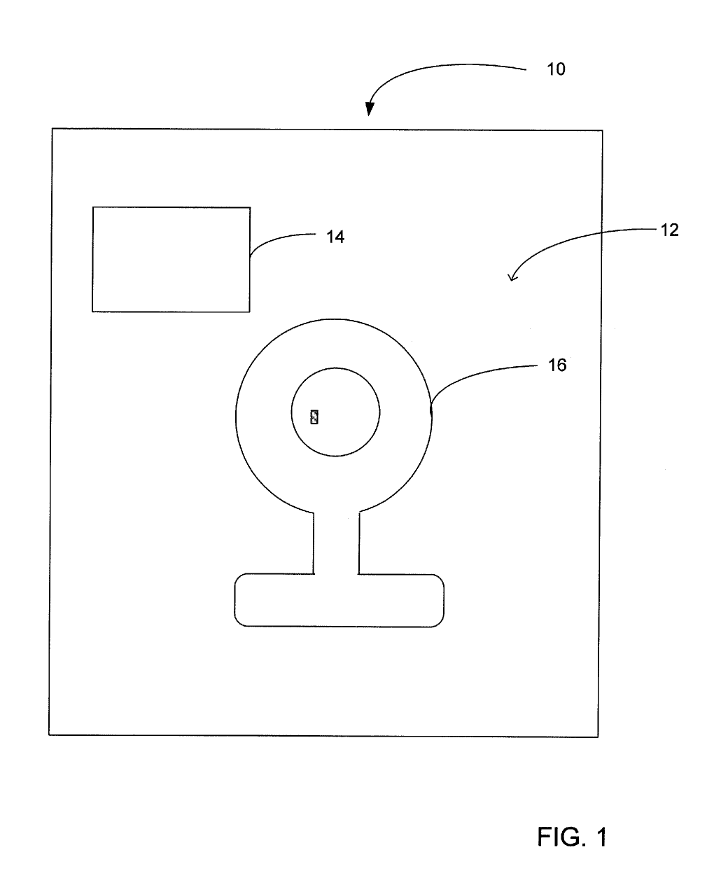

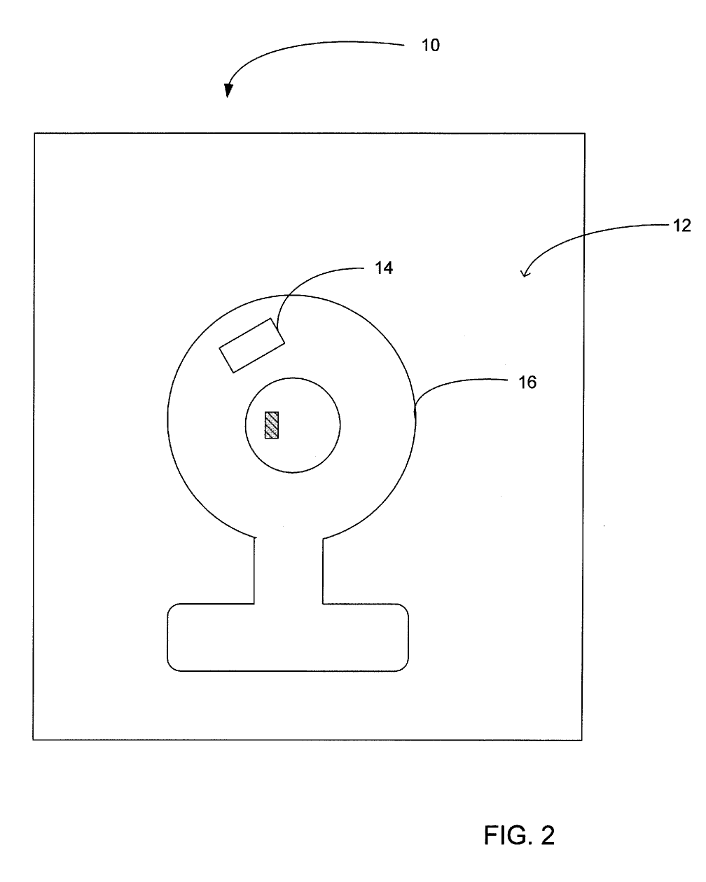

With reference to FIG. 1, the system of the invention is disclosed as element 10. A generally enclosed environment 12 is provided for the processing of food for humans and/or animals. In the environment 12 is provided an oxidative gas generator 14 such as a PHI Cell and food processing equipment such as a commercial freeze dryer 16. The oxidative gas generator 14 is installed in the environment 12 so that hydro peroxides, super oxide ions, ozonide ions, hydroxides and other oxidative gases are produced in proximity to the equipment. Equivalent generators of hydro peroxides, super oxide ions, ozonide ions, hydroxides and other oxidative gases as known in the art are also contemplated by this invention. The system, method and apparatus of the instant invention also contemplates the installation of an oxidative gas generator 14 on the surface of the equipment, as shown in FIG. 2 for a commercial freeze dryer 16.

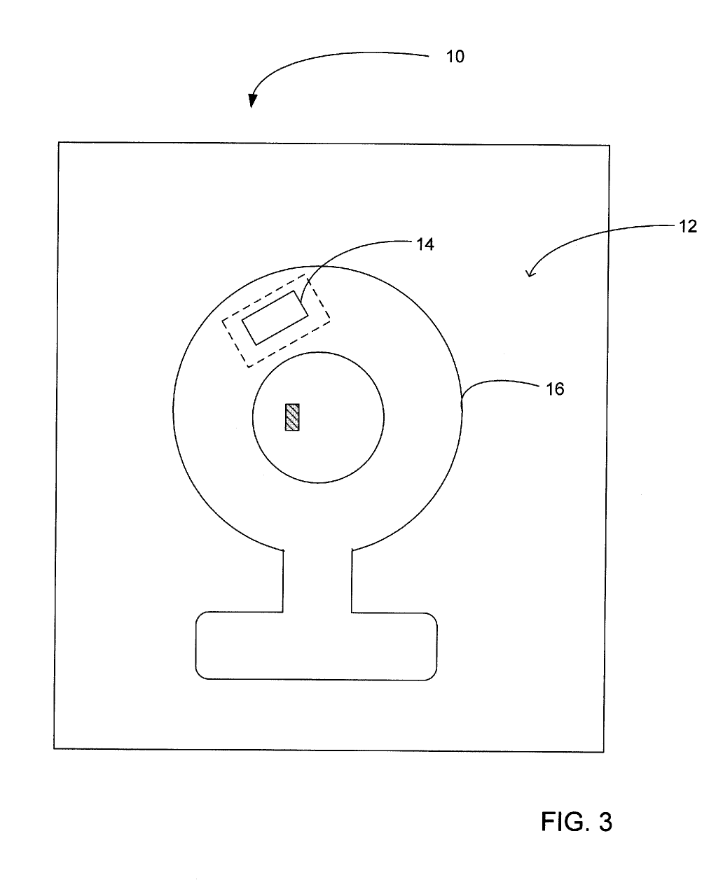

With reference to FIG. 3, the system of the invention is again shown as element 10. A generally enclosed environment 12 is provided for the processing of food. In this embodiment, an oxidative gas generator 14 is installed within the equipment. FIG. 2 shows the oxidative gas generator 14 located within the freeze drying area of a commercial freeze dryer 16.

It will be appreciated by persons skilled in the art that the present invention is not limited to what has been particularly shown and described herein above. In addition, unless mention was made above to the contrary, it should be noted that the accompanying drawings are not to scale. A variety of modifications and variations are possible in light of the above teachings without departing from the scope and spirit of the invention.

What is claimed is:

1. A system for controlling microbial contamination, comprising:
a generally enclosed environment for the processing of food;
a PHI Cell located within the enclosed environment; and
food processing equipment within the generally enclosed environment;
wherein low levels of oxidative gases are produced by the PHI Cell in proximity to the food processing equipment.

2. The system of claim 1, wherein the oxidative gases include hydro peroxides, super oxide ions, ozonide ions and hydroxides.

3. The system of claim 1, wherein the generally enclosed environment includes a Cleaned in Place (CIP) system.

4. The system of claim 1, wherein the PHI Cell is installed on the surface of the food processing equipment.

5. The system of claim 1, wherein the food processing equipment comprises a commercial freeze dryer.

6. The system of claim 1, wherein the PHI Cell is installed within the interior of the food processing equipment.

7. A device for controlling microbial contamination in a generally enclosed environment, comprising:
food processing equipment; and
a PHI Cell installed on the surface of the food processing equipment.

8. The device of claim 7, wherein the generally enclosed environment comprises a CIP system.

9. The device of claim 7, wherein the food processing equipment further comprises a commercial freeze dryer.

10. A device for controlling microbial contamination in a generally enclosed environment, comprising:
food processing equipment; and
a PHI Cell installed within the food processing equipment.

11. The device of claim 10, wherein the generally enclosed environment comprises a CIP system.

12. The device of claim 10, wherein the food processing equipment further comprises a commercial freeze dryer.

13. A method for controlling microbial contamination, comprising the steps of:
generally enclosing an environment;
locating a PHI Cell within the enclosed environment; and
placing food processing equipment within the generally enclosed environment such that low levels of oxidative gases generated by the PHI Cell are produced in proximity to the food processing equipment.

14. The method of claim 13, wherein the generally enclosed environment comprises a CIP system.

15. The method of claim 13, further comprising the steps of:
placing food in the generally enclosed environment; and
processing of food in the generally enclosed environment.

16. The method of claim 13, wherein the food processing equipment comprises a commercial food dryer.

17. The method of claim 13, wherein the PHI Cell is located on the surface of the food processing equipment.
